(19) United States
(12) Patent Application Publication
(10) Pub. No.: US 2023/0190463 A1
(43) Pub. Date: Jun. 22, 2023

(54) PROSTHETIC HEART VALVE INCLUDING AN EXPANSION AND LOCKING MECHANISM TRANSITIONABLE BETWEEN LOCKED AND UNLOCKED STATES

(71) Applicant: EDWARDS LIFESCIENCES CORPORATION, Irvine, CA (US)

(72) Inventor: Noam Nir, Pardes-Hanna (IL)

(21) Appl. No.: 18/110,705

(22) Filed: Feb. 16, 2023

Related U.S. Application Data
(63) Continuation of application No. PCT/US2021/046313, filed on Aug. 17, 2021.
(60) Provisional application No. 63/066,926, filed on Aug. 18, 2020.

(51) Int. Cl. A61F 2/24

(52) U.S. Cl. CPC A61F 2/243; A61F 2/2466

(57) ABSTRACT

An expansion and locking mechanism for a prosthetic valve and associated methods are disclosed. As one example, an expansion and locking mechanism for an implantable prosthetic device can include a first member coupled to a frame of the device and including at least one extension member extending in a circumferential direction and having a free end biased radially inward, into an interior of the first member and a second member coupled to the frame at a second location spaced apart from the first location, the second member extending at least partially inside the first member and comprising a plurality of radial extensions spaced apart from one another along a portion of a length of the second member, the plurality of radial extensions arranged in a first side of the second member that is arranged opposite a second side of the second member, the second side not including any radial extensions.

### CROSS REFERENCE TO RELATED APPLICATION

This application is a continuation of PCT Application No. PCT/US2021/046313, filed Aug. 17, 2021, which claims the benefit of U.S. Provisional Pat. Application No. 63/066,926, filed Aug. 18, 2020, which is incorporated herein by reference in its entirety.

### FIELD

The present disclosure relates to implantable, mechanically expandable prosthetic devices, such as prosthetic heart valves, and to methods and delivery assemblies for, and including, such devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters.

In some examples, mechanically expandable prosthetic heart valves can include expansion and locking mechanism that allow the prosthetic valve to be radially expanded to a desired diameter and then held (e.g., locked) in the desired diameter. Mechanically expandable prosthetic heart valves can also be compressed after an initial expansion (e.g., for repositioning and/or retrieval). However, compressing a locked prosthetic heart valve can be difficult and require additional release members.

Accordingly, a need exists for improved expansion and locking mechanisms for prosthetic heart valves.

### SUMMARY

Described herein are embodiments of improved prosthetic implant delivery assemblies and frames therefor, as well as related methods and devices for such assemblies. In several embodiments, the disclosed assemblies are configured for delivering replacement heart valves into a heart of a patient.

In one example, an implantable prosthetic device includes: a frame movable between a radially compressed and a radially expanded configuration, the frame comprising at least one expansion and locking mechanism. The at least one expansion and locking mechanism includes a first member coupled to the frame at a first location, the first member comprising at least one extension member extending in a circumferential direction and having a free end biased radially inward, into an interior of the first member, relative to a central longitudinal axis of the at least one expansion and locking mechanism. The at least one expansion and locking mechanism further includes a second member coupled to the frame at a second location spaced apart from the first location, the second member extending at least partially inside the first member and comprising a plurality of radial extensions spaced apart from one another along a portion of a length of the second member, where the plurality of radial extensions are arranged in a first side of the second member, the first side arranged opposite a second side of the second member, across the central longitudinal axis, the second side not including any radial extensions. Engagement of the at least one extension member with the plurality of radial extensions allows relative movement between the first member and the second member in a first axial direction to allow radial expansion of the frame and prevents relative movement in a second axial direction to prevent radial compression of the frame. The second member is rotatable within the first member into a first position where the at least one extension member is biased radially inward and engaged with the plurality of radial extensions and into a second position where the at least one extension member is pushed radially outward and in contact with the second side of the second member.

In some example, an assembly includes a prosthetic valve, the prosthetic valve including: a frame comprising a plurality of interconnected struts and movable between a radially compressed and radially expanded configuration and at least one expansion and locking mechanism. The expansion and locking mechanism includes: a first member coupled to the frame at a first location, the first member comprising at least one extension member extending in a circumferential direction and having a free end biased radially inward, into an interior of the first member, relative to a central longitudinal axis of the at least one expansion and locking mechanism. The expansion and locking mechanism further includes a second member coupled to the frame at a second location spaced apart from the first location, the second member extending at least partially into the first member and comprising a rack portion including a plurality of spaced apart radial extensions arranged in a first side of the rack portion, the first side arranged opposite a second side of the rack portion that is continuous with a remainder of the second member and does not include radial extensions. The assembly further includes a delivery apparatus comprising: a handle; a first actuation member extending from the handle and coupled to the first member, the first actuation member configured to apply a distally directed force to the first member; and a second actuation member extending from the handle and coupled to the second member, the second actuation member configured to apply a proximally directed force to the second member. The prosthetic valve is radially expandable from the radially compressed configuration to the radially expanded configuration upon application of the distally directed force and the proximally directed force to the prosthetic valve via the first and second actuation members, respectively. Expansion of the prosthetic valve causes movement of the first and second members relative to one another such that the at least one extension member engages the first side of the rack portion of the second member allowing movement of the first and second members in a first direction to allow radial expansion of the frame and preventing movement in a second direction to prevent radial compression of the frame. The prosthetic valve is radially compressible from the radially expanded configuration to a less expanded configuration upon rotation of the second member such that the at least one extension member is disengaged from the plurality of radial extensions and engages the second side of the rack portion and allows movement of the first and second members in the first direction and the second direction.

In some example, an implantable prosthetic device includes: a frame movable between a radially compressed and a radially expanded configuration, the frame comprising an inflow end portion and an outflow end portion. The device further includes at least one expansion and locking mechanism comprising: a first member coupled to the frame at a first location, the first member comprising a first extension member and a second extension member spaced apart from one another in an axial direction, each extending in a circumferential direction and having a free end biased radially inward, into an interior of the first member, relative to a central longitudinal axis of the at least one expansion and locking mechanism; and a second member coupled to the frame at a second location spaced apart from the first location, the second member extending at least partially inside the first member and comprising a plurality of radial extensions spaced apart from one another along a portion of a length of the second member, where the plurality of radial extensions are arranged in a first side of the second member, the first side arranged opposite a second side of the second member, across the central longitudinal axis, the second side not including any radial extensions. Engagement of the first and second extension members with the plurality of radial extensions allows relative movement between the first member and the second member in a first axial direction to allow radial expansion of the frame and prevents relative movement in a second axial direction to prevent radial compression of the frame. The second member is rotatable within the first member into a first position where the first and second extension members are engaged with the plurality of radial extensions and into a second position where the first and second extension members are pushed radially outward and engaged with the second side of the second member.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an example of a prosthetic heart valve.

FIG. 2 is a side view of another example of a prosthetic heart valve shown in a radially collapsed configuration.

FIG. 3 is a side view of the frame of the prosthetic heart valve of FIG. 2 shown in a radially expanded configuration.

FIG. 4 is a side elevation view of an example of a prosthetic valve delivery assembly.

FIG. 5 is a perspective view of a frame for a prosthetic heart valve comprising three expansion and locking mechanisms, according to some example.

FIG. 6 is a top plan view of the frame and expansion and locking mechanisms of FIG. 5.

FIG. 7 is a perspective view of an example of an expansion and locking mechanism of a prosthetic valve, the expansion and locking mechanism having a ratchet mechanism that is transitionable between a locking state and an unlocked state.

FIG. 8 is a cross-sectional side view of the expansion and locking mechanism of FIG. 7.

FIG. 9 is a perspective view of an inner member of the expansion and locking mechanism of FIG. 7.

FIG. 10 is a cross-sectional end view of an outer member of the expansion and locking mechanism of FIG. 7.

FIG. 11 is a cross-sectional side view of the expansion and locking mechanism of FIG. 7 showing a detail view of the ratchet mechanism in the locking state.

FIG. 12 is a cross-sectional end view of the expansion and locking mechanism of FIG. 7 showing the ratchet mechanism in the locking state.

FIG. 13 is a cross-sectional side view of the expansion and locking mechanism of FIG. 7 showing a detail view of the ratchet mechanism in the unlocked state.

FIG. 14 is a cross-sectional end view of the expansion and locking mechanism of FIG. 7 showing the ratchet mechanism in the unlocked state.

FIG. 15 is a cross-sectional side view of another example of an expansion and locking mechanism of a prosthetic valve, the expansion and locking mechanism having a ratchet mechanism that is transitionable between a locking state and an unlocked state and that includes multiple extension members.

FIG. 16 is a perspective view of the expansion and locking mechanism of FIG. 15 with the multiple extension members oriented in opposite circumferential directions.

FIG. 17 is a perspective view of the expansion and locking mechanism of FIG. 15 with the multiple extension members oriented in a same circumferential direction.

### DETAILED DESCRIPTION

### General Considerations

For purposes of this description, certain aspects, advantages, and novel features of the embodiments of this disclosure are described herein. The described methods, systems, and apparatus should not be construed as limiting in any way. Instead, the present disclosure is directed toward all novel and non-obvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Features, integers, characteristics, compounds, chemical moieties, or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, with reference to the prosthetic heart valve and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

### Examples of the Disclosed Technology

Described herein are examples of prosthetic implant delivery assemblies and components thereof which can improve a physician’s ability to control the size of a mechanically-expandable prosthetic implant, such as prosthetic valves (e.g., prosthetic heart valves or venous valves), stents, or grafts, as well as facilitate separation of the prosthetic implant from the delivery assembly, during the implantation procedure. The present disclosure also provides frames for use with such prosthetic implants. The frames can comprise locking mechanisms configured to hold the frame in an expanded configuration when the implant is expanded at a selected implantation site within a patient.

Prosthetic valves disclosed herein can be radially compressible and expandable between a radially compressed configuration and a radially expanded configuration. Thus, the prosthetic valves can be crimped on an implant delivery apparatus in the radially compressed configuration during delivery, and then expanded to the radially expanded configuration once the prosthetic valve reaches the implantation site.

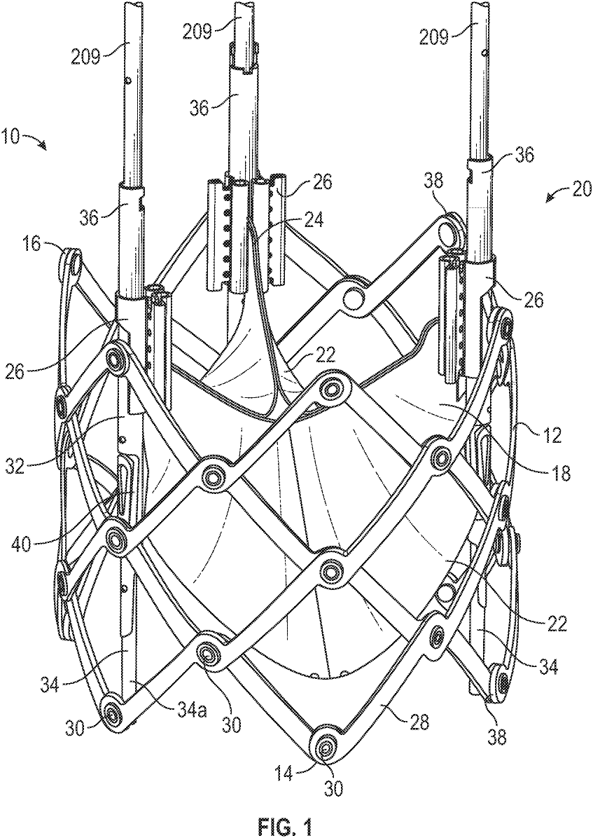

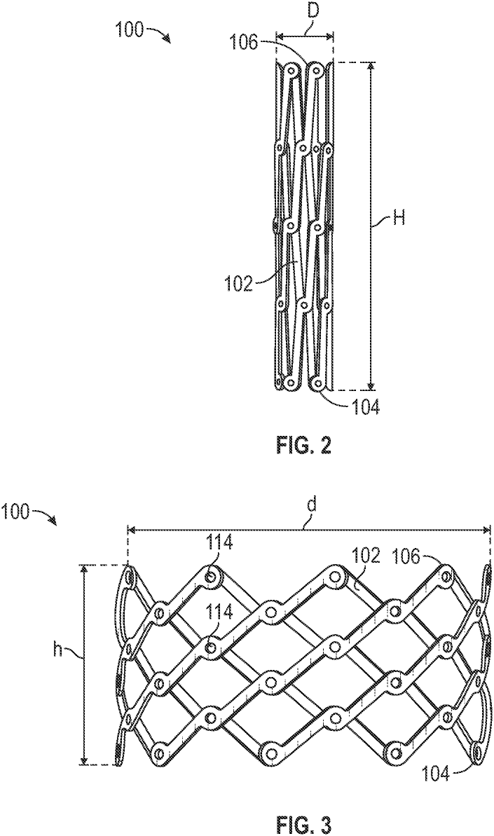

FIG. 1 shows an exemplary prosthetic valve 10, according to one example. The prosthetic valve 10 can be radially compressible and expandable between a radially compressed configuration for delivery into a patient (see e.g., FIG. 2) and a radially expanded configuration (see e.g., FIGS. 1 and 3). Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 10 can include an annular stent or frame 12 having a first end 14 and a second end 16. In the depicted examples, the first end 14 is an inflow end and the second end 16 is an outflow end. The outflow end 16 can be coupled to a delivery apparatus for delivering and implanting the prosthetic valve within the native aortic valve is a transfemoral, retrograde delivery approach. Thus, in the delivery configuration of the prosthetic valve, the outflow end 16 is the proximal-most end of the prosthetic valve. In some examples, the inflow end 14 can be coupled to the delivery apparatus, depending on the particular native valve being replaced and the delivery technique that is used (e.g., transseptal, transapical, etc.). For example, the inflow end 14 can be coupled to the delivery apparatus (and therefore is the proximal-most end of the prosthetic valve in the delivery configuration) when delivering the prosthetic valve to the native mitral valve via a transseptal delivery approach.

The prosthetic valve 10 can also include a valvular structure 18 which is coupled to the frame 12 and configured to regulate the flow of blood through the prosthetic valve 10 from the inflow end to the outflow end. The prosthetic valve 10 can further include a plurality of actuators 20 mounted to and equally spaced around the inner surface of the frame 12. Each of the actuators 20 can be configured to form a releasable connection with one or more respective actuators of a delivery apparatus, as further described below.

The valvular structure 18 can include, for example, a leaflet assembly comprising one or more leaflets 22 (three leaflets 22 in the illustrated example) made of a flexible material. The leaflets 22 of the leaflet assembly can be made from in whole or part, biological material, bio-compatible synthetic materials, or other such materials. Suitable biological material can include, for example, bovine pericardium (or pericardium from other sources). The leaflets 22 can be arranged to form commissures 24, which can be, for example, mounted to respective actuators 20. Further details regarding transcatheter prosthetic heart valves, including the manner in which the valvular structure can be coupled to the frame 12 of the prosthetic valve 10, can be found, for example, in U.S. Pat. Nos. 6,730,118, 7,393,360, 7,510,575, 7,993,394, and 8,652,202, and U.S. Pat. Application Publication No. 2018/0325665, all of which are incorporated herein by reference in their entireties.

In some examples, the prosthetic valve 10 can include a plurality of commissure support elements configured as commissure clasps or clamps 26. In the illustrated configuration, the prosthetic valve includes a commissure clamp 26 positioned at each commissure 24 and configured to grip adjacent portions of two leaflets 22 at each commissure 24 at a location spaced radially inwardly of the frame 12. Each clamp 26 can be mounted on an actuator 20 as shown. In some examples, the commissure supports elements (such as clamps 26) can be mounted to the struts 28 of the frame, or alternatively, the commissures 24 can be mounted (e.g., sutured) directly to the struts of the frame. Further details of the commissure clamps 26 and other techniques for mounting the commissures of a valve assembly to a frame can be found in U.S. Pat. Application Publication No. 2018/0325665.

Although not shown, the prosthetic valve 10 can also include one or more skirts or sealing members. For example, the prosthetic valve 10 can include an inner skirt mounted on the inner surface of the frame. The inner skirt can function as a sealing member to prevent or decrease perivalvular leakage, to anchor the leaflets 22 to the frame, and/or to protect the leaflets against damage caused by contact with the frame during crimping and during working cycles of the prosthetic valve. The prosthetic valve 10 can also include an outer skirt mounted on the outer surface of the frame 12. The outer skirt can function as a sealing member for the prosthetic valve by sealing against the tissue of the native valve annulus and helping to reduce paravalvular leakage past the prosthetic valve. The inner and outer skirts can be formed from any of various suitable biocompatible materials, including any of various synthetic materials (e.g., PET) or natural tissue (e.g., pericardial tissue). The inner and outer skirts can be mounted to the frame using sutures, an adhesive, welding, and/or other means for attaching the skirts to the frame.

The frame 12 can be made of any of various suitable materials, such as stainless steel, a cobalt chromium alloy, or a nickel titanium alloy (“NiTi”), for example Nitinol. Referring again to FIG. 1, as shown, the frame 12 can include a plurality of interconnected struts 28 arranged in a lattice-type pattern. The struts 28 are shown as positioned diagonally, or offset at an angle relative to, and radially offset from, a longitudinal axis of the prosthetic valve 10 when the prosthetic valve 10 is in the expanded configuration. In other implementations, the struts 28 can be offset by a different amount than depicted in FIG. 1, or some or all of the struts 28 can be positioned parallel to the longitudinal axis of the prosthetic valve 10.

In the illustrated example, the struts 28 are pivotably coupled to one another at one or more pivot joints or junctions along the length of each strut. For example, in the illustrated configuration, each of the struts 28 can be formed with apertures (see e.g., apertures 114 in FIG. 3) at opposing ends of the strut and apertures spaced along the length of the strut. Respective hinges can be formed at the locations where struts 28 overlap each other via fasteners or pivot members, such as rivets or pins 30 that extend through the apertures. The hinges can allow the struts 28 to pivot relative to one another as the frame 12 is radially expanded or compressed, such as during assembly, preparation, or implantation of the prosthetic valve 10.

In some examples, the frame 12 can be constructed by forming individual components (e.g., the struts and fasteners of the frame) and then mechanically assembling and connecting the individual components together. In some examples, the struts 28 are not coupled to each other with respective hinges but are otherwise pivotable or bendable relative to each other to permit radial expansion and contraction of the frame 12. For example, the frame 12 can be formed (e.g., via laser cutting, electroforming or physical vapor deposition) from a single piece of material (e.g., a metal tube). Further details regarding the construction of the frame and the prosthetic valve are described in U.S. Publication Nos. 2018/0153689; 2018/0344456; 2019/0060057 all of which are incorporated herein by reference. Additional examples of expandable prosthetic valves that can be used with the delivery apparatuses disclosed herein are described in U.S. Publication No. 2015/0135506 and 2014/0296962, which are incorporated herein by reference.

Referring still to FIG. 1, in some examples, the prosthetic valve 10 can comprise one or more actuators 20 configured to produce radial expansion and compression of the frame. The one or more actuators in the illustrated example comprise one or more push-pull mechanisms 32 coupled to the frame 12. In the illustrated example, the prosthetic valve 10 has three push-pull mechanisms 32, however, in some example a greater or fewer number of push-pull mechanisms 32 can be used.

Each push-pull mechanism 32 can generally comprise an inner member 34, such as an inner tubular member, and an outer member 36 disposed about the inner member 34. The inner members 34 and the outer members 36 can be movable longitudinally relative to each other in a telescoping manner to radially expand and contract the frame 12, as further described in U.S. Publication Nos. 2018/0153689, 2018/0153689 and 2018/0325665 which are incorporated herein by reference. The inner members 34 can be, for example, rods, cables, wires, or tubes. The outer members 36 can be, for example, tubes or sheaths having sufficient rigidity such that they can apply a distally directed force to the frame without bending or buckling.

The inner members 34 can have distal end portions 34a coupled to the inflow end 14 of the frame 12 (e.g., with a coupling element such as a pin 30). In the illustrated example, each of the inner members 34 are coupled to the frame at respective apices 38 at the inflow end 14 of the frame 12. For example, the distal end portion 34a of each inner member 34 can be pivotably connected to the rivet or pin 30 that connects the two struts at the adjacent apex 38. The outer members 36 can be coupled to apices 38 at the outflow end 16 of the frame 12 at, for example, a mid-portion of the outer member 36, as shown in FIG. 1, or at a proximal end portion of the outer member, as desired. The outer members 36 can be pivotably connected to the rivet or pin 30 that connects the two struts at the adjacent apex 38.

The inner member 34 and the outer member 36 can telescope relative to each other between a fully contracted state (corresponding to a fully radially expanded state of the prosthetic valve) and a fully extended state (corresponding to a fully radially compressed state of the prosthetic valve). In the fully extended state, the inner member 34 is fully extended from the outer member 36. In this manner, the push-pull mechanisms 32 allow the prosthetic valve to be fully expanded or partially expanded to different diameters and retain the prosthetic valve in the partially or fully expanded state. It should be understood that the inner members 34 and the outer members 36 can be coupled to other locations on the frame to produce radial compression and expansion of the frame, so long as the inner member and outer member of each actuator are coupled at axial spaced pivot joints of the frame.

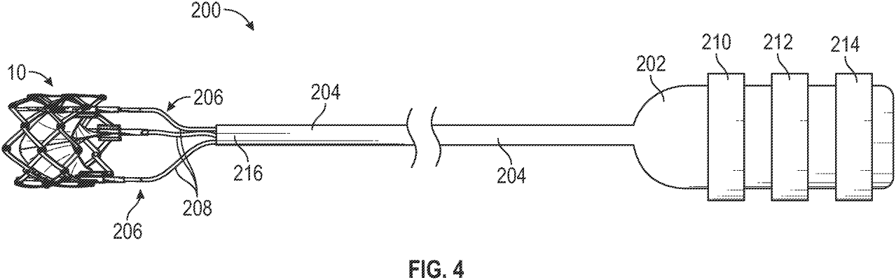

In use, a delivery apparatus (see, e.g., delivery apparatus 200 in FIG. 4) can be releasably coupled to the actuators 20 (e.g., push-pull mechanisms 32) of prosthetic valve 10. For example, the delivery apparatus can have one or more actuator assemblies (e.g., actuator assemblies 206 in FIG. 4) that are releasably coupled to respective actuators 20 (e.g., push-pull mechanisms 32) of the prosthetic valve. The actuator assemblies can be configured to transfer expansion forces (e.g., pushing and/or pulling forces) from a handle of the delivery apparatus to the push-pull mechanisms 32 of the prosthetic valve. Each of the actuator assemblies can include an inner or second actuation member 209 (as shown in FIG. 1) that is releasably coupled to a respective inner member 34 of a push-pull mechanism 32. Each actuator assembly of the delivery apparatus can also include an outer member 208 (FIG. 4) that is releasably coupled to a respective outer member 36 of a push-pull mechanism 32.

Once coupled to the delivery apparatus, the prosthetic valve 10 can then be radially collapsed (see e.g., FIG. 2) and the distal end portion of the delivery apparatus, along with the radially collapsed valve, can be inserted into a patient. Once the prosthetic valve 10 is at the desired implantation site, the prosthetic valve can be radially expanded (see e.g., FIG. 3). In some examples, as shown in FIG. 1, the push-pull mechanisms 32 can comprise one or more locking mechanisms 40, allowing the frame 12 to maintain an expanded diameter after the prosthetic valve is released from the delivery apparatus. Additional details of the locking mechanism 40 can be found in Pat. Publication No. 2018/0153689.

FIGS. 2-3 illustrate a bare frame 102 (without the leaflets and other components) of another exemplary example of a prosthetic valve 100 for purposes of illustrating expansion of the prosthetic valve from the radially compressed configuration to the radially expanded configuration. FIG. 2 shows the frame 102 in the radially compressed configuration, and FIG. 3 shows the frame 102 in the fully radially expanded configuration. In the radially compressed configuration, the frame 102 has a first height, H, and a first diameter, D. In the radially expanded configuration, the frame 102 has a shorter, second height, h, and a larger, second diameter, d. The prosthetic valve 100 in the illustrated configuration can be radially expanded by maintaining the first end 104 of the frame 102 at a fixed position while applying a force in the axial direction against the second end 106 toward the first end 104. Alternatively, the prosthetic valve 100 can be expanded by applying an axial force against the first end 104 while maintaining the second end 106 at a fixed position, or by applying opposing axial forces to the first and second ends 104, 106, respectively.

FIG. 4 illustrates a delivery apparatus 200, according to one example, adapted to deliver a prosthetic heart valve, such as the illustrated prosthetic heart valve 10, described above. The prosthetic valve 10 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 202, a first elongated shaft 204 (which comprises an outer shaft in the illustrated example) extending distally from the handle 202, at least one actuator assembly 206 (e.g., three in the illustrated example) extending distally through the outer shaft 204. The at least one actuator assembly 206 can be configured to radially expand and/or radially collapse the prosthetic valve 10 when actuated. Though the illustrated example shows three actuator assemblies 206, it should be understood that more or fewer actuator assemblies can be present. In some examples, a distal end portion 216 of the shaft 204 can be sized to house the prosthetic valve in its radially compressed, delivery state during delivery of the prosthetic valve through the patient’s vasculature. In this manner, the distal end portion 216 functions as a delivery sheath or capsule for the prosthetic valve during delivery,

The actuator assemblies 206 can be releasably coupled to the prosthetic valve 10. For example, in the illustrated example, each actuator assembly 206 can be coupled to a respective actuator 20 of the prosthetic valve 10. Each actuator assembly 206 can comprise a first actuation member 208 and a second actuation member 209 (FIG. 1) extending through the first actuation member 208. When actuated, the first and second actuation members 208, 209 can move axially relative to one another to transmit pushing and/or pulling forces to portions of the prosthetic valve. The actuator assemblies 206 can be at least partially disposed radially within, and extend axially through, one or more lumens of the outer shaft 204. For example, the actuator assemblies 206 can extend through a central lumen of the shaft 204 or through separate respective lumens formed in the shaft 204. In the illustrated example, the second actuation member (not shown) extends through the first actuation member 208. However, in other embodiments, the first and second actuation members may be spaced apart from each other circumferentially around the prosthetic valve 10.

The first actuation member 208 can be, for example, a sleeve, cylinder, shaft, tube, or other member configured to apply a distally directed forced to the prosthetic valve. The second actuation member 209 (FIG. 1) can comprise an elongated actuator member in the form of, for example, a rod, shaft, cable, wire, suture, or other member configured to apply a proximally directed force to the prosthetic valve.

As mentioned above, each actuator 20 of the prosthetic valve 10 can generally comprise an inner member 34 and an outer member 36 disposed about the inner member 34. The inner members 34 and the outer members 36 can be movable longitudinally relative to each other in a telescoping manner to radially expand and contract the frame 12. In some examples, each first actuation member 208 of the delivery apparatus 200 can be releasably coupled to a respective outer member 36 of the actuator 20, and each second actuation member 209 can be releasably coupled to a respective inner member 34 of the actuator 20. A user can actuate the actuator assemblies (e.g., using knob 212 as described below) thereby causing axial movement of the first actuation member 208 relative to the second actuation member. Movement of the actuator assemblies 206 can result in corresponding movement of the actuators 20 to radially expand and/or collapse the frame 12. Once the prosthetic valve 10 is fully expanded, it can be locked into position using one or more locking mechanisms and/or locking features, as described in more detail below.

The handle 202 of the delivery apparatus 200 can include one or more control mechanisms (e.g., knobs or other actuating mechanisms) for controlling different components of the delivery apparatus 200 in order to expand and/or deploy the prosthetic valve 10. For example, in the illustrated example the handle 202 comprises first, second, and third knobs 210, 212, and 214.

The first knob 210 can be a rotatable knob configured to produce axial movement of the outer shaft 204 relative to the prosthetic valve 10 in the distal and/or proximal directions in order to deploy the prosthetic valve from the delivery sheath 216 once the prosthetic valve has been advanced to a location at or adjacent the desired implantation location with the patient’s body. For example, rotation of the first knob 210 in a first direction (e.g., clockwise) can retract the outer shaft 204 proximally relative to the prosthetic valve 10 and rotation of the first knob 210 in a second direction (e.g., counter-clockwise) can advance the outer shaft 204 distally. In some examples, the first knob 210 can be actuated by sliding or moving the knob 210 axially, such as pulling and/or pushing the knob. In some examples, actuation of the first knob 210 (rotation or sliding movement of the knob 210) can produce axial movement of the actuator assemblies 206 (and therefore the prosthetic valve) relative to the delivery sheath 216.

The second knob 212 can be a rotatable knob configured to produce radial expansion and/or contraction of the prosthetic valve 10. For example, rotation of the second knob 212 can move the first and second actuation members 208, 209 axially relative to one another. Rotation of the second knob 212 in a first direction (e.g., clockwise) can radially expand the prosthetic valve 10 and rotation of the second knob 212 in a second direction (e.g., counter-clockwise) can radially collapse the prosthetic valve 10. In some examples, the second knob 212 can be actuated by sliding or moving the knob 212 axially, such as pulling and/or pushing the knob.

The third knob 214 can be a rotatable knob configured to release the prosthetic heart valve 10 from the delivery apparatus 200. For example, rotation of the third knob in a first direction (e.g., clockwise) can disengage the actuator assemblies 206 from the actuators 20 of the prosthetic valve 10. In some examples, the third knob 214 can be actuated by sliding or moving the third knob 214 axially, such as pulling and/or pushing the knob.

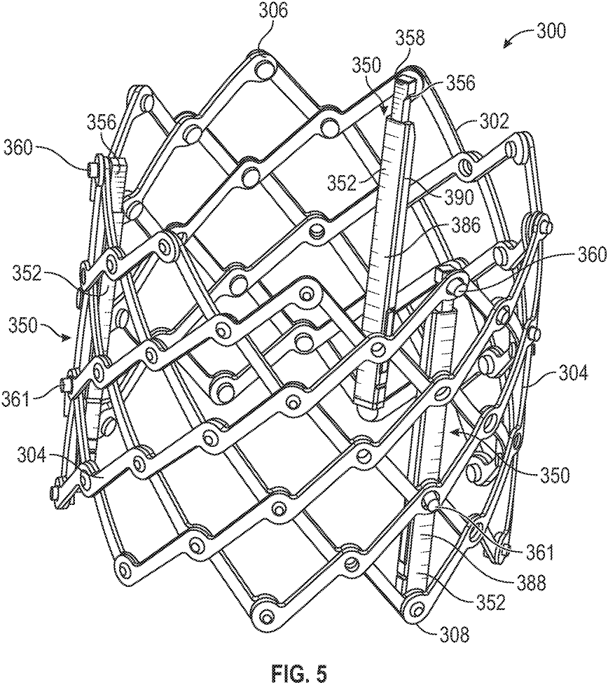

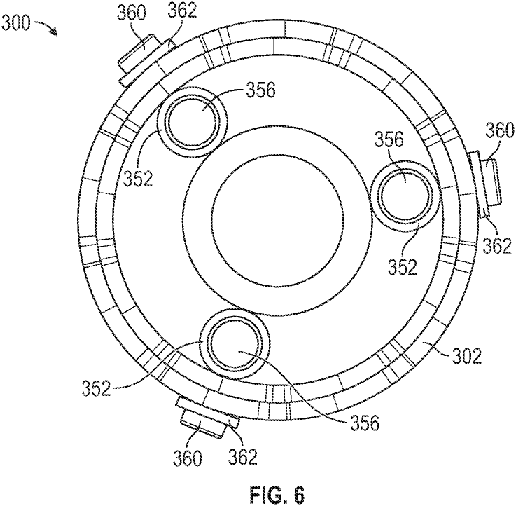

FIGS. 5 and 6 illustrate an exemplary example of a prosthetic valve 300, according to another example, the prosthetic valve 300 comprising a frame 302 and one or more expansion and locking mechanisms 350. The frame 302 comprises a plurality of pivotably connected struts 304 defining an inflow end 306 (which is the distal end of the frame in a delivery configuration for the illustrated example) and an outflow end 308 (which is the proximal end of the frame in the delivery configuration for the illustrated example). The struts 304 are pivotably connected to each other at a plurality of junctions that permit pivoting of the struts relative to each other when the frame 302 is radially compressed and expanded, as described above in connection with prosthetic valve 10.

The prosthetic valve 300 can include a valvular structure (e.g., valvular structure 18) and inner and/or outer skirts, as previously described, although these components are omitted from FIGS. 5 and 6 for purposes of illustration. The one or more expansion and locking mechanisms 350 can be used in lieu of or in addition to actuators 20 described above with reference to FIG. 1. The expansion and locking mechanisms 350 can be used to both radially expand and lock the frame 302 of prosthetic valve 300 in a radially expanded state. In some examples, the commissures of the leaflets may be attached to a commissure support portion of the expansion and locking mechanisms 350. In some examples, the commissures of the leaflets may be attached to additional commissure posts of the frame 302.

FIGS. 5 and 6 show three expansion and locking mechanisms 350 mounted to the frame 302 with the frame 302 shown in the radially expanded configuration. Though the illustrated example shows three expansion and locking mechanisms 350 spaced apart from each other about the circumference of the frame, it should be noted that a prosthetic valve can comprise any number of expansion and locking mechanisms 350. For example, in some examples, a prosthetic valve can comprise a single expansion and locking mechanism, or two expansion and locking mechanisms, or four expansion and locking mechanisms, etc. The expansion and locking mechanisms 350 can be placed at any position about the circumference of the frame 302. For example, in some examples, such as the illustrated example, the expansion and locking mechanisms 350 are equally spaced from one another about the circumference of the frame 302. In some examples, it can be advantageous to have two or more expansion and locking mechanisms situated adjacent to one another.

Each expansion and locking mechanism 350 can include an outer member in the form of a sleeve 352 having an inner lumen, cavity, or bore and an inner member 356 extending at least partially into the cavity.

In some examples, as shown in FIG. 5, the sleeve 352 comprises an inner wall 386, an outer wall 388, and two side walls 390, each of which extends radially between a longitudinal edge of the inner wall 386 and an opposing longitudinal edge of the outer wall 388. The inner wall 386, the outer wall 388, and the two side walls 390 define the cavity, which is sized and shaped to receive the inner member 356.

The sleeve 352 in the illustrated example of FIG. 5 has a rectangular shape in cross-section and the inner member 356 has a rectangular shape in cross-section corresponding to the shape of the bore. In some examples, the sleeve 352 and/or the inner member 356 can have a square cross-sectional profile or various other corresponding shapes in cross-section, for example, circular, ovular, triangular, rectangular, square, or combinations thereof.

In some examples, as shown in FIG. 6, the sleeve 352 and the inner member 356 can have circular cross-sections. As such, in some examples, the sleeve 352 and the sleeve 352 can be concentric cylinders.

As best shown in FIG. 5, a distal end portion 358 of the inner member 356 can be coupled to the frame 302 at a first location via a fastener 360 that is affixed to and extends radially from the distal end portion 358 of the inner member 356. The fastener 360 can be for example, a rivet or pin. As shown, in some examples, the fastener 360 can extend through corresponding apertures at a junction of two overlapping struts 304 of the frame 302 and can serve as a pivot pin around which the two struts 304 can pivot relative to each other and the inner member 356. In some examples, an end cap or nut 362 (as shown in FIG. 6) can be disposed over an end portion of the fastener 360. The nut 362 can have a diameter greater than the diameter of the apertures to retain the fastener 360 within the apertures. In some examples, the inner member 356 need not comprise a fastener 360 and can be coupled to the frame 302 via other means of attachment such as welding, adhesives, etc.

The sleeve 352 can be coupled to the frame 302 at a second location, axially spaced from the first location. For example, in the illustrated example, the inner member 356 is secured to the frame 302 near the distal or inflow end 306 of the frame and the sleeve 352 is secured to the frame 302 closer to or at the proximal or outflow end 308 of the frame, such as via a fastener 361 (e.g., a rivet or pint). The fastener 361 is affixed to and extends radially from the sleeve 352 through corresponding apertures at a junction of two overlapping struts 304 and can serve as a pivot pin around which the two struts 304 can pivot relative to each other and the sleeve 352. A nut 362 can be mounted on each fastener 361 to retain the fastener within the corresponding apertures. The expansion and locking mechanism 350 can be pivotably coupled to the frame 302 at any two axially spaced, circumferentially aligned locations on the frame.

The inner member 356 can be axially movable relative to the sleeve 352 in a proximal direction and in a distal direction, along a central longitudinal axis of the frame 302. As such, because the inner member 356 and the sleeve 352 are secured to the frame at axially spaced locations, moving the inner member 356 and the sleeve 352 axially with respect to one another in a telescoping manner can cause radial expansion or compression of the frame 302. For example, moving the inner member 356 proximally toward the outflow end 308 of the frame, while holding the sleeve 352 in a fixed position and/or moving the sleeve 352 distally toward the inflow end 306 of the frame can cause the frame 302 to foreshorten axially and expand radially. Conversely, moving the inner member 356 distally and/or moving the sleeve 352 proximally causes the frame 302 to elongate axially and compress radially.

A prosthetic valve 300 including one or more expansion and locking mechanisms 350 can be expanded in the following exemplary manner. Generally, the prosthetic valve 300 is placed in a radially compressed state and releasably coupled to a distal end portion of a delivery apparatus (e.g., delivery apparatus 200 of FIG. 4), and then advanced through the vasculature of a patient to a selected implantation site (e.g., the native aortic annulus). The prosthetic valve 300 can then be deployed at the implantation site and expanded and locked in the expanded configuration using the expansion and locking mechanisms 350. Further details regarding the prosthetic valve, the expansion and locking mechanisms, and delivery apparatuses for actuating the expansion and locking mechanism can be found in U.S. Provisional Application Nos. 62/928,291 and 62/950,005, the contents of which are incorporated herein by reference. Additionally, further details regarding alternate examples of expansion and locking mechanisms, including various examples of a locking or ratcheting mechanism, can be found in U.S. Provisional Application Nos. 62/928,291, 62/950,005, 62/981,666, 63/013,912, and 63/026,267, the contents of which are incorporated herein by reference.

In some examples, expansion and locking mechanisms for a prosthetic valve, such as one of the expansion and locking mechanisms described above, can include a ratcheting mechanism or assembly that is configured to lock the prosthetic valve in position, as it is radially expanded with the expansion and locking mechanisms. In this way, the prosthetic valve can be held in a plurality of radially expanded positions, as determined by the ratcheting mechanism, during expansion, thereby preventing the prosthetic valve from compressing back down to a more radially compressed configuration. Further, the expansion and locking mechanism can lock the prosthetic valve in an expanded state after reaching the desired expansion diameter. However, with such ratcheting mechanisms, once the prosthetic valve is in a locked position, unlocking the prosthetic valve to allow for radial compression of the prosthetic valve may not be possible or may require the utilization of additional release members. Further, such release members may be difficult to operate and/or may add components and manufacturing costs to the overall assembly. Thus, it is desirable to provide an expansion and locking mechanism that includes a ratcheting mechanism for locking the prosthetic valve in various radially expanded positions and that provides an additional unlocking capability with structural components that are easier to manufacture (thereby reducing manufacturing costs).

FIGS. 7-16 show examples of expansion and locking mechanisms for a prosthetic valve having a ratchet mechanism or assembly that is configured to transition between a locking state and unlocked state for radial expansion and compression of a prosthetic valve to which they are coupled. In some examples, the expansion and locking mechanisms have circular cross-sections and can be used on any of the prosthetic valves described herein for radially expanding (and/or compressing) and locking the prosthetic valve in a desired configuration. The expansion and locking mechanisms presented in FIGS. 7-16 can be configured to radially expand the prosthetic valve to which they are attached, to operate in the locking state wherein the prosthetic valve is prevented from being radially compressed, as it is being radially expanded, and switch from the locking state to the unlocked state where the prosthetic valve can be freely radially compressed and/or radially expanded. In some examples, the expansion and locking mechanisms of FIGS. 7-16 can replace the expansion and locking mechanisms 350 of FIGS. 5 and 6.

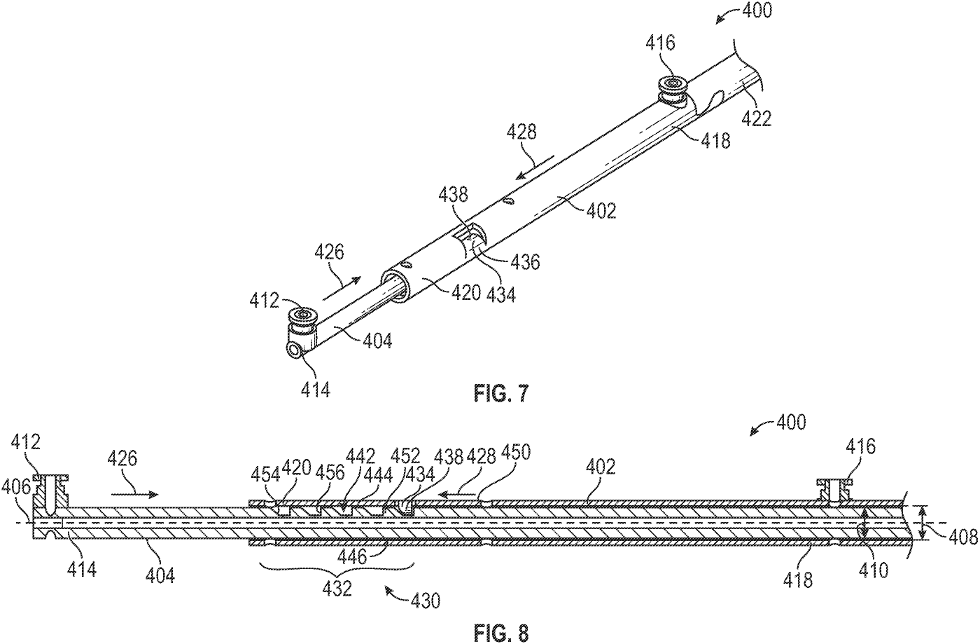

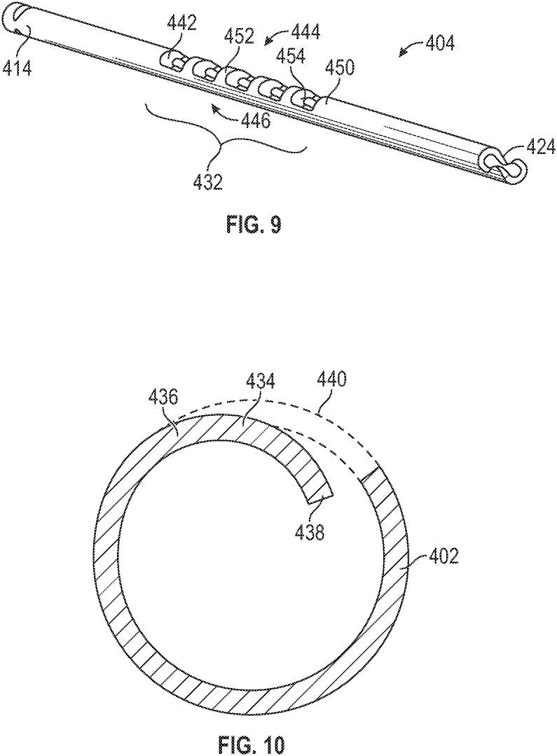

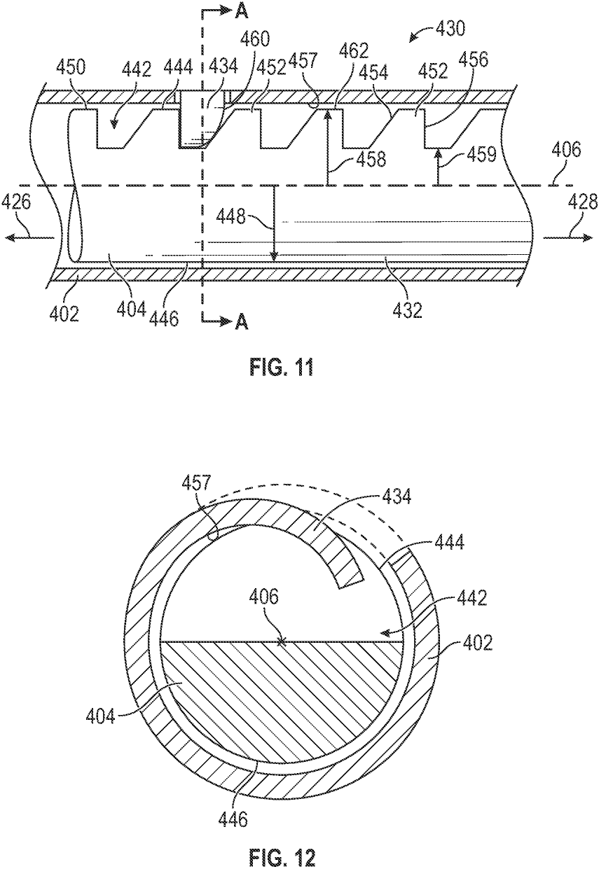

Turning first to FIGS. 7-14, a first example of an expansion and locking mechanism 400 having a ratchet mechanism that is transitionable between a locking state and an unlocked state is shown. FIG. 7 shows a perspective view of the expansion and locking mechanism 400 which comprises an outer member 402 (which may also be referred to herein as an outer housing or sleeve) and an inner member 404 (which may also be referred to herein as an inner rack), the inner member 404 arranged at least partially inside the outer member 402. FIG. 8 shows a cross-sectional side view of the expansion and locking mechanism 400 and FIG. 11 shows a detail view of the cross-sectional side view of the expansion and locking mechanism 400. FIG. 9 shows a perspective view of the inner member 404, removed from the outer member 402 and FIG. 10 shows a cross-sectional end view of the outer member 402, at a region of an extension member 434 of the outer member 402.

As shown in FIGS. 7 and 8, the outer member 402 is formed as a tubular member, thereby allowing the inner member 404 to extend therethrough and move axially (relative to a central longitudinal axis 406 of the expansion and locking mechanism 400) within the outer member 402. For example, an inner diameter 408 of the outer member 402 can be larger than an outer diameter 410 of the inner member 404, thereby allowing the inner member 404 to move axially and rotate within the outer member 402 (as described further below). As shown in FIGS. 7 and 8, the inner member 404 extends outward from a first (e.g., distal) end 420 of the outer member 402. The outer member 402 can be rotationally fixed.

In some examples, as shown in FIGS. 7 and 8, the expansion and locking mechanism 400 can include a first fastener (e.g., hinge) 412 coupled to a first (e.g., distal) end 414 of the inner member 404 and a second fastener (e.g., hinge) 416 coupled to a second (e.g., proximal) end 418 of the outer member 402. In some examples, similar to as shown in FIGS. 5 and 6, the first fastener 412 can be configured to couple the inner member 404 to an inflow end (e.g., inflow end 306 shown in FIG. 5) of a frame of a prosthetic valve and the second fastener 416 can be configured to couple the outer member 402 to an outflow end (e.g., outflow end 308 shown in FIG. 5) of the frame. However, in some examples, alternate coupling arrangements of the fasteners 412 and 416 is possible, as described above.

As shown in FIG. 7, in some examples, an outer member 422 of an actuator mechanism of a delivery apparatus (e.g., similar to outer member 208 shown in FIG. 4) can be coupled to the second end 418 of the outer member 402. Further, as introduced above, an inner member of the actuator mechanism (e.g., second or inner actuation member 209 shown in FIG. 1) can be coupled to a second (e.g., proximal) end 424 of the inner member 404 (FIG. 9).

As introduced above, by pulling the inner member 404 and the outer member 402 closer together (e.g., via moving the first end 414 of the inner member 404 and the first end 420 of the outer member 402 closer together), the prosthetic valve frame to which they are coupled can be radially expanded (e.g., during implantation). In some examples, as shown in FIGS. 7 and 8, the inner member 404 is configured to translate axially, in a first direction 426 while the outer member 402 is either held stationary or translated axially, in a second direction 428, in order to radially expand the prosthetic valve frame. In some examples, the outer member 402 can be configured to translate axially in the second direction 428 while the inner member 404 is held stationary to radially expand the prosthetic valve frame.

The expansion and locking mechanism 400 can include a ratchet mechanism 430 (FIG. 8) formed by a portion (e.g., rack portion) 432 of the inner member 404 (FIGS. 8 and 9) and at least one extension member 434 (FIGS. 7, 8, and 10) on the outer member 402 which are configured to selectively interface with one another. As explained further below, the ratchet mechanism 430 can be configured to transition between the locking state and the unlocked state. In the locking state, the ratchet mechanism 430 can be configured to allow axial translation of the inner member 404 relative to the outer member 402 in a first direction (e.g., to radially expand the frame) while preventing axial translation of the inner member 404 relative to the outer member 402 in an opposite, second direction (thereby preventing radially compression of the frame). In the unlocked state, the ratchet mechanism 430 can be further configured to allow the inner member 404 and the outer member 402 to move freely in the axial direction relative to one another, thereby allowing both radial expansion and radial compression of the prosthetic valve frame (without locking it in position).

As shown in FIGS. 7, 8, and 10, the outer member 402 includes the extension member 434 which extends in a circumferential direction and radially inward into an interior of the outer member 402. For example, FIG. 10 includes a dashed line 440 indicating a path of the extension member 434 if it were not biased radially inward, as shown, and continued along the circumference of the outer member 402, with a remainder of the outer member 402. Instead, the extension member 434 bends radially inward and away from the dashed line 440.

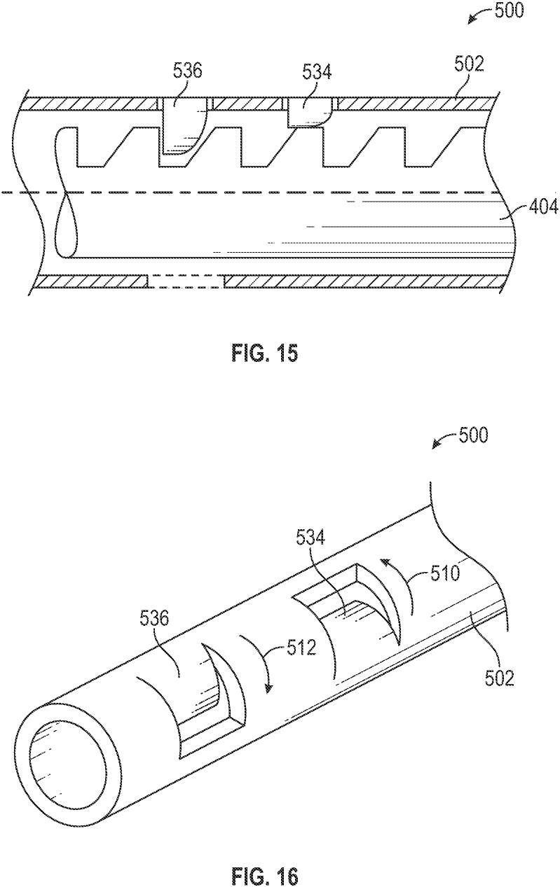

While only a single extension member 434 is shown in the example of FIGS. 6-14, in some examples, the outer member 402 can include more than one extension member 434 (such as two, three, four, or the like). For example, as described further below with reference to FIGS. 15 and 16, in some examples, the outer member 402 can include two extension members.

In some examples, the extension member 434 is arranged closer to the first end 420 than the second end 418 of the outer member 402 (FIGS. 7 and 8).

The extension member 434 can be integrally formed with a remainder of the outer member 402. For example, the extension member 434 can include a first, attached end 436 that is continuous with the remainder of the outer member 402 and a second, free end 438 that is unattached (FIGS. 7 and 10). The extension member 434, between its attached end 436 and free end 438, is orientated in the circumferential direction (e.g., extends in a direction along a circumference of the outer member 402).

The extension member 434 can extend only over a portion of the circumference of the outer member 402. For example, as shown in FIGS. 7 and 10 (and FIGS. 12 and 14, as described further below), the circumferentially extending extension member 434 can span around less than 180 degrees of the outer member 402. Thus, the portion of the outer member 402 in which the extension member 434 is arranged can include a ratcheting side, which includes the extension member 434, and an opposing, smooth side which does not include an extension member 434 (as shown in FIGS. 8 and 10).

The free end 348 of the extension member 434 can be biased radially inward, into the interior of the outer member 402 and toward the inner member 404. For example, in some examples, the free end 348 of the extension member 434 can be preset radially inward and can be configured to flex back toward the outer surface of the outer member 402, in response to a radially outward force applied to an inner surface of the free end 348 (as explained further below).

For example, the extension member 434 can be spring-biased in the radially inward orientation shown in FIG. 10. In some examples, one or more of a material, thickness, and/or setting (e.g., shape set via heat setting or another known method) of the extension member 434 can provide the above-described preset orientation and flexibility or spring-like configuration of the extension member 434.

In some examples, the extension member 434 can be more easily shape set in the radially inwardly biased configuration due to is extending in the circumferential direction (as opposed to the axial direction).

In some examples, the outer member 402 can be a laser cut tube and the at least one extension member 434 can be laser cut into the tube and then shape set into the preset, radially inwardly biased orientation (e.g., as shown in FIGS. 10 and 11).

As shown in FIGS. 8 and 9, the inner member 404 includes the rack portion 432 which includes a plurality of radial depressions 442 arranged in a first, ratcheting side 444 of the rack portion 432. A second, smooth side 446 of the rack portion 432, which is arranged opposite (around the circumference) from the ratcheting side, does not include any radial depressions and has a radius 448 that is continuous along the rack portion 432 and a remainder of the inner member 404 (shown in FIG. 11, as described further below). In some examples, the radial depressions 442 can be referred to as radially extending spaces or apertures that are formed in the inner member 404.

In some examples, the radial depressions 442 can be cut-outs that are cut into the outer surface 450 of the inner member (on the ratcheting side 444) via laser cutting or another cutting method.

The radial depressions 442 can extend radially inward, into the inner member 404, from the outer surface 450 of the ratcheting side 444 of the inner member 404. The radial depressions 442 form a plurality of spaced apart radial extensions 452 in the ratcheting side 444 of the rack portion 432. In some examples, the radial extensions 452 are configured as teeth. For example, each radial extension 452 is formed between two adjacent radial depressions 442. As such, the radial extensions are axially spaced apart from one another along the ratcheting side 444 of the inner member 404.

The radial extensions 452 can each have a radius 458 that is approximately the same as the radius 448 of the smooth side 446 of the rack portion while a radius 459 of each trough of the radial depressions 442 is smaller that the radius 448 and the radius 458 (FIG. 11)

In some examples, the radius 448 can be the same or similar to the radius 459 and the radial extensions 452 can extend outward from an outer surface of the inner member 404. In this way, the radial depressions 442 can be formed by the radial extensions 452 (e.g., between adjacent radial extensions 452).

As shown in FIGS. 8, 9, and 11, the radial depressions 442 and corresponding radial extensions 452 can be ramped in one direction (e.g., toward the first end 414 of the inner member 404, as shown in FIG. 8). For example, as best seen in FIG. 11, each radial extension 452 can have an angled or slanted edge 454 and an opposing, relatively perpendicular (or less angled) edge 456.

In some examples, the angled edge 454 can be angled at an angle that is in a range of about 30 to about 80 degrees, about 40 to about 75 degrees, or about 45 to about 70 degrees from a line parallel to the central longitudinal axis 406. In some examples, the relatively perpendicular edge 456 is arranged at an angle that is in a range of about85 to about 95 degrees from the line parallel to the central longitudinal axis 406. In some examples, the relatively perpendicular edge 456 is arranged at an angle of approximately 90 degrees from the line parallel to the central longitudinal axis 406.

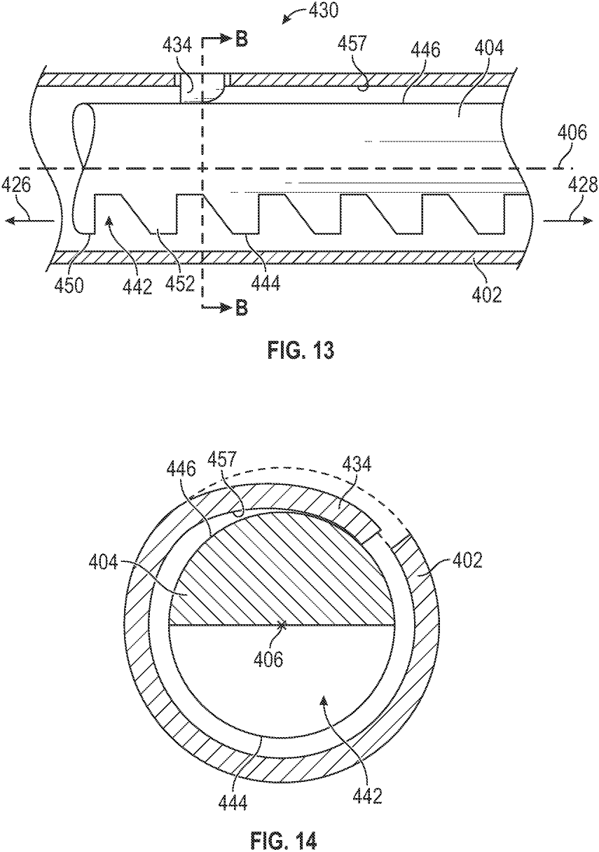

Together, the rack portion 432 of the inner member 404 and the at least one extension member 434 of the outer member 402 form the ratchet mechanism 430. As introduced above, the radial extensions 452, formed by the radial depressions 442, and the extension member 434 are configured to selectively engage and interface with one another. For example, the inner member 404 is rotatable, around the central longitudinal axis 406, within an interior (e.g., inner lumen) of the outer member 402. By rotating the inner member 404 while the outer member 402 is held stationary in the circumferential direction, the ratchet mechanism 430 can be transitioned between a locking state and an unlocked state. FIGS. 11 and 12 are cross-sectional side and end views, respectively, of the ratchet mechanism 430 in the locking state (e.g., configuration) and FIGS. 13 and 14 are cross-sectional side and end views, respectively, of the ratchet mechanism 430 in the unlocked state. FIG. 12 is taken along Section A-A in FIG. 11 and FIG. 14 is taken along Section B-B in FIG. 13.

As shown in FIGS. 11 and 12, the inner member 404 is rotated such that the ratcheting side 444 of the rack portion 432 is positioned adjacent to a first side 457 of the outer member 402 that includes the extension member 434, and thus, the radial extensions 452 are engaged with the extension member 434. In this configuration, the radial extensions 452 can function as ratcheting teeth which may engage with the extension member 434 (which may function as a pawl), as the inner member 404 and outer member 402 move axially relative to one another.

The angled edges 454 of the radial extensions 452 enable the rack portion 432 of the inner member 404 to slide relatively smoothly over the extension member 434 of the outer member 402 (e.g., as the inner member translates axially in the first direction 426, relative to the outer member 402, to radially expand the frame of the prosthetic valve).

In some examples, the extension member 434 can have a corresponding angled, curved, or slanted edge 460 (FIG. 11) that further facilitates the sliding engagement between the extension member 434 and the angled edges 454 of the radial extensions 452.

For example, as the inner member 404 moves axially in the first direction 426 (FIG. 11) relative to the outer member 402, the extension member 434 slides along the angled edge 454 of one of the radial extensions 452, and as a result, is compressed radially toward an inner surface of the outer member 402, until the extension member 434 reaches a crest (e.g., peak) 462 of the radial extension 452 (such a position is shown in FIG. 15, as described further below). As the inner member 404 moves further axially in the first direction, the extension member 434 can slide off the crest 462 of the radial extension 452 and into one of the radial depressions 442, thereby extending back into is radially inwardly biased configuration (e.g., closer to the central longitudinal axis 406), as shown in FIGS. 11 and 12. For example, FIG. 12 shows the extension member 434 extending radially inward, toward the central longitudinal axis 406, within the radial depression 442.

When the extension member 434 is arranged within one of the radial depressions 442, between two adjacent radial extensions 452, as shown in FIGS. 11 and 12, the frame of the prosthetic valve is locked in the current, radially expanded configuration and is prevented from being radially recompressed. For example, if the inner member 404 is moved axially in the second direction 428, relative to the outer member 402, the relatively perpendicular edge 456 of the radial extension 452 arranged adjacent to a relatively perpendicular, second edge (e.g., the edge opposite the first edge 460) of the extension member 434 stops the extension member 434 from moving over the radial extensions 452. As a result, the prosthetic valve is locked in the selected expanded diameter.

As shown in FIGS. 13 and 14, the inner member 404 is rotated such that the smooth side 446 of the rack portion 432 is positioned adjacent to the first side 457 of the outer member, and thus, the extension member 434 is engaged with the relatively smooth side 446 of the inner member 404 which does not include the radial extensions 452. In this configuration, as shown in FIG. 14, the extension member 434 is pushed more radially outward, toward the inner surface of the outer member 402, by the smooth side 446 of the inner member 404. Since the smooth side 446 of the inner member 404 does not include any radial extensions 452, the inner member 404 and the outer member 402 can translate freely, in the first direction 426 and the second direction 428. As such, this is referred to herein as the unlocked state of the ratchet mechanism 430. In this state, the prosthetic valve can be radially recompressed (e.g., after an initial radial expansion) and or radially expanded (without the locking functionality described above when in the locking state).

For example, in some examples, prosthetic valve recompression may be desired for re-crossing or repositioning at the implantation site. In this situation, the inner member 404 can be rotated, approximately 180 degrees, from the locking state shown in FIGS. 11 and 12 to the unlocked state shown in FIGS. 13 and 14. The inner member 404 can then translate freely, proximally and distally, thereby enabling valve recompression when it is translated in the second direction 428. The prosthetic valve can then be re-expanded and locked by rotating the inner member 404 back into the position shown in FIGS. 11 and 12 (e.g., the locking state), where the ratcheting side is arranged adjacent to the extension member 434.

In some examples, rotation of the inner member 404 can be actuated by an actuator (e.g., a user-controllable mechanism) in a handle of the delivery apparatus (such as handle 202 shown in FIG. 4). For example, in some examples, the handle 202 of delivery apparatus 200 can include an additional knob or button that controls rotation of the inner member 404 relative to the outer member 402.

FIGS. 15-17 show a second example of an expansion and locking mechanism 500 having a ratchet mechanism that is transitionable between a locking state and an unlocked state, including the inner member 404 and an outer member 502. The outer member 502 may be the same as the outer member 402 of FIGS. 7, 8, and 10-14, except it can include two or more extension members (which may be the same or similar to extension members 434 shown in FIGS. 7, 8, and 10-14). Two or more extension members may increase the expansion resolution of the expansion and locking mechanism 500. For example, the expansion and locking mechanism 500 may have more locking positions along its length due to the two extension members (as compared to only one extension member of the expansion and locking mechanism 400).

As shown in FIGS. 15-17, the outer member 502 includes a first extension member 534 and a second extension member 536 which are spaced apart from one another along a central longitudinal axis of the expansion and locking mechanism 500.

In a first example, as shown in FIG. 16, the first extension member 534 and the second extension member 536 can be angularly phased relative to each other, such that each of the extension members 534 and 536 extends radially inward from an opposite circumferential direction. For example, a circumferential extension direction 510 of the first extension member 534 is opposite to a circumferential extension direction 512 of the second extension member 536 (e.g., the free ends of each of the first extension member 534 and the second extension member 536 are arranged circumferentially opposite one another).

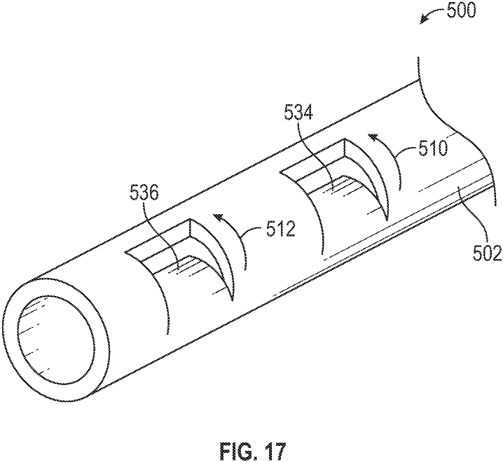

In a second example, as shown in FIG. 17, the first extension member 534 and the second extension member 536 can be oriented in a same circumferential direction such that each of the extension members 534 and 536 extends radially inward from a same circumferential direction. For example, the circumferential extension direction 510 of the first extension member 534 is the same as the circumferential extension direction 512 of the second extension member 536 (e.g., the free ends of each of the first extension member 534 and the second extension member 536 are oriented the same way in the example of FIG. 17.

In some examples, the outer member 502 can include more than two extension members, such as three, four, or the like.

The above-described expansion and locking mechanisms (e.g., expansion and locking mechanisms 400 and 500) may be relatively simple to operate, including allowing for easy transitioning between locked and unlocked states (e.g., via rotation of the inner member) to enable re-compression or the prosthetic valve if re-crossing or re-positioning is required during an implantation procedure. Moreover, the expansion and locking mechanisms including the ratcheting mechanism transitionable between the locking and unlocked states may include a smaller number of components that are relatively easy to manufacture, thereby simplifying the overall manufacturing process for the expansion and locking mechanism and reducing costs. For example, in some examples, the ratchet mechanism described herein can be formed by employing tube laser-cutting and simplified shape-setting processes, which may be more simplified and cost-effective manufacturing methods.

### Delivery Techniques

For implanting a prosthetic valve (e.g., prosthetic valve 10) within the native aortic valve via a transfemoral delivery approach, the prosthetic valve is mounted in a radially compressed state along the distal end portion of a delivery apparatus (e.g., delivery apparatus 200). The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable prosthetic device, comprising: a frame movable between a radially compressed and a radially expanded configuration; and at least one expansion and locking mechanism comprising: a first member coupled to the frame at a first location, the first member comprising at least one extension member extending in a circumferential direction and having a free end biased radially inward, into an interior of the first member, relative to a central longitudinal axis of the at least one expansion and locking mechanism; and a second member coupled to the frame at a second location spaced apart from the first location, the second member extending at least partially inside the first member and comprising a plurality of radial extensions spaced apart from one another along a portion of a length of the second member, wherein the plurality of radial extensions are arranged in a first side of the second member, the first side arranged opposite a second side of the second member, across the central longitudinal axis, the second side not including any radial extensions, wherein engagement of the at least one extension member with the plurality of radial extensions allows relative movement between the first member and the second member in a first axial direction to allow radial expansion of the frame and prevents relative movement in a second axial direction to prevent radial compression of the frame, and wherein the second member is rotatable within the first member into a first position where the at least one extension member is biased radially inward and engaged with the plurality of radial extensions and into a second position where the at least one extension member is pushed radially outward and in contact with the second side of the second member.

Example 2. The implantable prosthetic device of any example herein, particularly example 1, wherein the plurality of radial extensions are formed by a plurality of radial depressions that extend into an outer surface of the second member, on the first side of the second member.

Example 3. The implantable prosthetic device of any example herein, particularly example 2, wherein a radius of the second side of the second member and a radius of each radial extension of the plurality of radial extensions is the same.

Example 4. The implantable prosthetic device of any example herein, particularly example 2 or example 3, wherein the plurality of radial depressions is formed by cuts into the outer surface, on the first side of the portion of the second member including the plurality of radial extensions.

Example 5. The implantable prosthetic device of any example herein, particularly any one of examples 1-4, wherein in the second position the at least one extension member is disengaged from the plurality of radial extensions and engaged with the second side of the second member to allow relative movement between the first member and the second member in the first axial direction and the second axial direction.

Example 6. The implantable prosthetic device of any example herein, particularly any one of examples 1-5, wherein each radial extension of the plurality of radial extensions has an angled edge that is angled relative to a line perpendicular to the central longitudinal axis and an opposing edge that is not angled relative to the line.

Example 7. The implantable prosthetic device of any example herein, particularly example 6, wherein the at least one extension member has a corresponding angled edge that is configured to interface with and slide along the angled edge of each radial extension.

Example 8. The implantable prosthetic device of any example herein, particularly any one of examples 1-7, wherein the first and second members have a circular cross-sectional profile in a plane normal to the central longitudinal axis of the expansion and locking mechanism.

Example 9. The implantable prosthetic device of any example herein, particularly any one of examples 1-8, wherein the at least one extension member is shape set into a radially inwardly biased orientation where the free end extends toward the central longitudinal axis and away from an inner surface of the first member.

Example 10. The implantable prosthetic device of any example herein, particularly any one of examples 1-9, wherein an entirety of the portion of the length of the second member including the plurality of radial extensions is enclosed within the first member.

Example 11. The implantable prosthetic device of any example herein, particularly any one of examples 1-10, wherein the frame comprises an inflow end portion and an outflow end portion, and wherein the first location is the outflow end portion and the second location is the inflow end portion.

Example 12. The implantable prosthetic device of any example herein, particularly any one of examples 1-11, wherein the at least one extension member is a first extension member of the first member, wherein the first member further includes a second extension member spaced axially apart from the first extension member.

Example 13. An assembly, comprising: a prosthetic valve comprising: a frame comprising a plurality of interconnected struts and movable between a radially compressed and radially expanded configuration; and at least one expansion and locking mechanism comprising: a first member coupled to the frame at a first location, the first member comprising at least one extension member extending in a circumferential direction and having a free end biased radially inward, into an interior of the first member, relative to a central longitudinal axis of the at least one expansion and locking mechanism; and a second member coupled to the frame at a second location spaced apart from the first location, the second member extending at least partially into the first member and comprising a rack portion including a plurality of spaced apart radial extensions arranged in a first side of the rack portion, the first side arranged opposite a second side of the rack portion that is continuous with a remainder of the second member and does not include radial extensions; and a delivery apparatus comprising: a handle; a first actuation member extending from the handle and coupled to the first member, the first actuation member configured to apply a distally directed force to the first member; and a second actuation member extending from the handle and coupled to the second member, the second actuation member configured to apply a proximally directed force to the second member, wherein the prosthetic valve is radially expandable from the radially compressed configuration to the radially expanded configuration upon application of the distally directed force and the proximally directed force to the prosthetic valve via the first and second actuation members, respectively, wherein expansion of the prosthetic valve causes movement of the first and second members relative to one another such that the at least one extension member engages the first side of the rack portion of the second member, allowing movement of the first and second members in a first direction to allow radial expansion of the frame and preventing movement in a second direction to prevent radial compression of the frame, and wherein the prosthetic valve is radially compressible from the radially expanded configuration to a less expanded configuration upon rotation of the second member such that the at least one extension member is disengaged from the plurality of radial extensions and engages the second side of the rack portion and allows movement of the first and second members in the first direction and the second direction.

Example 14. The assembly of any example herein, particularly example 13, wherein the second member is configured to rotate within the first member and wherein the first member and the second member have circular cross-sections in a plan normal to the central longitudinal axis.

Example 15. The assembly of any example herein, particularly example 13 or example 14, wherein the first member and the second member are concentric with one another and the rack portion of the second member is arranged entirely within the first member.

Example 16. The assembly of any example herein, particularly any one of examples 13-15, wherein the plurality of radial extensions are formed by a plurality of radial depressions in the first side of the rack portion, the radial depressions spaced apart from one another along the rack portion of the second member.

Example 17. The assembly of any example herein, particularly example 16, wherein a crest of each radial extension of the plurality of radial extensions is arranged in line, in a radial direction, with an outer surface of a remaining portion of the second member, the remaining portion arranged on either side of the rack portion.

Example 18. The assembly of any example herein, particularly any one of examples 13-17, wherein each radial extension of the plurality of radial extensions includes an angled side and a non or less angled side, relative to a line arranged perpendicular to the central longitudinal axis, wherein the angled side and the non or less angled side are arranged on opposite sides of a crest of the radial extension.

Example 19. The assembly of any example herein, particularly example 18, wherein, during expansion of the prosthetic valve, when the first side of the rack portion is arranged adjacent to and engaged with the at least one extension member, the at least one extension member is configured to slide along the angled side of each radial extension and into a radial depression arranged between two adjacent radial extensions.

Example 20. The assembly of any example herein, particularly example 19, wherein the at least one extension member has a corresponding angled edge that is configured to interface with and slide along the angled side of each radial extension.

Example 21. The assembly of any example herein, particularly any one of examples 13-20, wherein the first location is an outflow end portion of the frame and the second location is an inflow end portion of the frame.

Example 22. The assembly of any example herein, particularly any one of examples 13-21, wherein the at least one extension member is a first extension member of the first member, wherein the first member further includes a second extension member spaced apart from the first extension member, and wherein the first extension member extends in a first circumferential direction and the second extension member extends in an opposite, second circumferential direction.

Example 23. The assembly of any example herein, particularly any one of examples 13-22, wherein the at least one extension member is integral with and formed into the first member and shape set into a radially inwardly biased orientation where the free end extends toward the central longitudinal axis and away from an inner surface of the first member.

Example 24. The assembly of any example herein, particularly any one of examples 13-23, wherein the at least one expansion and locking mechanism includes a first fastener configured to extend through first corresponding apertures at a junction of two overlapping struts of the frame and couple the first member to the first location of the frame and a second fastener configured to extend through second corresponding apertures at a junction of two overlapping struts of the frame and couple the second member to the second location of the frame.

Example 25. The assembly of any example herein, particularly any one of examples 13-24, wherein the prosthetic valve includes three of the expansion and locking mechanism spaced apart from one another around a circumference of the frame.

Example 26. The assembly of any example herein, particularly any one of examples 13-25, wherein the handle includes a control mechanism, actuatable by a user, that actuates relative movement of the first actuation member and the second actuation member.

Example 27. A method, comprising: axially translating a first member and a second member of an expansion and locking mechanism of a frame of a prosthetic valve relative to one another in only a first direction to radially expand the frame of the prosthetic valve and preventing axial translation of the first and second members relative to one another in a second direction to prevent radial compression of the frame via a plurality of radial extensions of the second member interfacing with at least one extension member of the first member, wherein the plurality of radial extensions are spaced apart from one another along a rack portion of the second member, on a first side of the rack portion, the rack portion including an opposite, second side that has a relatively smooth surface and does not include any radial extensions, wherein at least a portion of the second member, including the rack portion, is arranged within the first member, and wherein the at least one extension member extends circumferentially along the first member and has a free end that is biased radially inward, into an interior of the first member and toward the second member; rotating the second member within the first member to disengage the plurality of radial extensions from the at least one extension member and engage the second side of the rack portion with the at least one extension member; and while the at least one extension member is engaged with the second side of the rack portion, axially translating the first and second members relative to one another, freely in the second direction to radial compress the frame of the prosthetic valve.

Example 28. The method of any example herein, particularly example 27, wherein axially translating the first and second members relative to one another while the at least one extension member is engaged with the second side of the rack portion further comprises axially translating the first and second members relative to one another, freely in the first direction to radially expand the frame without locking the frame at a selected radially expanded diameter.

Example 29. The method of any example herein, particularly example 28, further comprising rotating the second member within the first member so that the first side of the rack portion is arranged adjacent to a side of the first member including the at least one extension member and the plurality of radial extensions are engaged with the at least one extension member to enable locking of the frame at the selected radially expanded diameter.

Example 30. The method of any example herein, particularly any one of examples 27-29, wherein the first direction is toward one another and second direction is away from one another.

Example 31. The method of any example herein, particularly any one of examples 27-30, wherein rotating the second member within the first member includes rotating the second member 180 degrees relative to the first member.

Example 32. The method of any example herein, particularly example 31, wherein rotating the second member within the first member by 180 degrees includes transitioning the expansion and locking mechanism between a locking state and an unlocked state.

Example 33. The method of any example herein, particularly any one of examples 27-32, wherein rotating the second member within the first member to disengage the plurality of radial extensions from the at least one extension member and engage the second side of the rack portion with the at least one extension member includes bending the at least one extension member radially outward, toward an inner surface of the first member.

Example 34. The method of any example herein, particularly example 33, wherein the axially translating the first and second members relative to one another, freely in the second direction to radial compress the frame of the prosthetic valve includes sliding the free end of the at least extension member along the relatively smooth surface of the second side of the rack portion of the second member.

Example 35. The method of any example herein, particularly any one of examples 27-34, wherein axially translating the first member and the second member relative to one another in only the first direction to radially expand the frame and preventing axial translation of the first and second members relative to one another in the second direction to prevent radial compression of the frame includes sliding the at least one extension member over angled edges of the plurality of radial extensions and into radial depressions arranged between the radial extensions, wherein for each radial extension, the angled edge is arranged opposite a non-angled edge of the radial extension.

Example 36. A method, comprising: inserting a distal end portion of a delivery apparatus into vasculature of a patient and advancing the distal end portion to a target implantation site, the delivery apparatus releasably coupled to a prosthetic valve movable between a radially compressed configuration and a radially expanded configuration, the prosthetic valve comprising a frame and at least one expansion and locking mechanism coupled to the frame, the expansion and locking mechanism including an inner member and outer member, the inner member arranged at least partially inside the outer member; radially expanding the prosthetic valve and locking the prosthetic valve at a selected radially expanded diameter and preventing recompression of the prosthetic valve during the expanding via a ratchet mechanism arranged in a locking state, the ratchet mechanism formed by a rack portion of the inner member and an extension member of the outer member, the rack portion including a first side with a plurality of spaced apart radial extensions arranged therein and a second side not including any radial extensions, the extension member extending in a circumferential direction and having a free end biased radially inward, into an interior of the outer member, wherein in the locking state the extension member is engaged with the plurality of radial extensions; rotating the inner member, relative to the outer member, to disengage the extension member from the plurality of radial extensions and transition the ratchet mechanism into an unlocked state where the extension member interfaces with the second side of the rack portion of the inner member; and while the ratchet mechanism is in the unlocked state, translating, in an axial direction, the inner member and the outer member freely relative to one another to radially expand or radially compress the prosthetic valve, without locking the prosthetic valve at the selected radially expanded diameter.

Example 37. The method of any example herein, particularly example 36, wherein translating the inner member and the outer member freely relative to one another includes recompressing the prosthetic valve from the selected radially expanded diameter that was selected prior to the rotating.

Example 38. The method of any example herein, particularly example 37, further comprising, after recompressing the prosthetic valve, rotating the inner member, relative to the outer member, to transition the ratchet mechanism into the locking state and engage the extension member with the plurality of radial extensions.

Example 39. The method of any of any example herein, particularly any one of examples 36-38, wherein the rotating includes rotating the inner member within the outer member and wherein the outer member is fixed from rotating.

Example 40. The method of any of any example herein, particularly any one of examples 36-39, wherein radially expanding the prosthetic valve includes moving ends of the inner member and the outer member that are configured to coupled to the frame towards one another, in an axial direction that is arranged along a central longitudinal axis of the expansion and locking mechanism.

Example 41. The method of any example herein, particularly example 40, wherein radially expanding the prosthetic valve includes sliding the extension member over angled edges of the plurality of radial extensions and into radial depressions arranged between the radial extensions, wherein for each radial extension, the angled edge is arranged opposite a non-angled edge of the radial extension, as the inner member and the outer member are moved towards one another.

Example 42. The method of any example herein, particularly any one of examples 36-41, wherein rotating the inner member to transition the ratchet mechanism into the unlocked state, from the locking state, includes rotating the inner member by 180 degrees within the outer member.

Example 43. The method of any example herein, particularly any one of examples 36-42, wherein the plurality of radial extensions are formed by a plurality of radial depressions that extend into an outer surface of the inner member, on the first side of the inner member, wherein each radial extension is formed between two adjacent radial depressions.

Example 44. The method of any example herein, particularly example 43, wherein a radius of the second side of the inner member and a radius of each radial extension of the plurality of radial extensions is the same.

Example 45. The method of any example herein, particularly any one of examples 36-44, wherein the inner member and the outer member have circular cross-sectional profiles in a plane normal to a central longitudinal axis of the expansion and locking mechanism.

Example 46. The method of any example herein, particularly any one of examples 36-45, wherein the extension member is shape set into a radially inwardly biased orientation where the free end extends toward a central longitudinal axis of the expansion and locking mechanism and away from an inner surface of the outer member and wherein rotating the inner member to transition the ratchet mechanism into the unlocked state includes pushing the free end of the extension member, with the second side of the rack portion of the inner member, toward the inner surface of the outer member.

Example 47. The method of any example herein, particularly any one of examples 36-46, wherein the inner member is coupled to an inflow end of the frame of the prosthetic valve and the outer member is coupled to an outflow end of the frame of the prosthetic valve.

48. The method of any example herein, particularly any one of examples 36-47, wherein the frame includes three of the expansion and locking mechanisms.

Example 49. An implantable prosthetic device, comprising: a frame movable between a radially compressed and a radially expanded configuration, the frame comprising an inflow end portion and an outflow end portion; and at least one expansion and locking mechanism comprising: a first member coupled to the frame at a first location, the first member comprising a first extension member and a second extension member spaced apart from one another in an axial direction, each extending in a circumferential direction and having a free end biased radially inward, into an interior of the first member, relative to a central longitudinal axis of the at least one expansion and locking mechanism; and a second member coupled to the frame at a second location spaced apart from the first location, the second member extending at least partially inside the first member and comprising a plurality of radial extensions spaced apart from one another along a portion of a length of the second member, wherein the plurality of radial extensions are arranged in a first side of the second member, the first side arranged opposite a second side of the second member, across the central longitudinal axis, the second side not including any radial extensions, wherein engagement of the first and second extension members with the plurality of radial extensions allows relative movement between the first member and the second member in a first axial direction to allow radial expansion of the frame and prevents relative movement in a second axial direction to prevent radial compression of the frame, and wherein the second member is rotatable within the first member into a first position where the first and second extension members are engaged with the plurality of radial extensions and into a second position where the first and second extension members are pushed radially outward and engaged with the second side of the second member.

Example 50. The implantable prosthetic device of any example herein, particularly example 49, wherein the first extension member and the second extension member are aligned with one another in the circumferential direction, on a same side of the first member.

Example 51. The implantable prosthetic device of any example herein, particularly example 49 or example 50, wherein the plurality of radial extensions is formed by a plurality of radial depressions that extend into an outer surface of the second member, on the first side of the second member.

Example 52. The implantable prosthetic device of any example herein, particularly example 51, wherein the first extension member and the second extension member are axially spaced apart from one another along the first member such that, when one of the first and second extension members is arranged in a radial depression of the plurality of radial depressions, another one of the first and second extension members is engaged with a crest of a radial extension of the plurality of radial extensions.

Example 53. The implantable prosthetic device of any example herein, particularly any one of examples 49-52, wherein each radial extension of the plurality of radial extensions has an angled edge that is angled relative to a line perpendicular to the central longitudinal axis and an opposing edge that is not angled relative to the line.

Example 54. The implantable prosthetic device of any example herein, particularly example 53, wherein the first and second extension members each have a corresponding angled edge that is configured to interface with and slide along the angled edge of each radial extension.

Example 55. The implantable prosthetic device of any example herein, particularly any one of examples 49-54, wherein the first and second members have a circular cross-sectional profile in a plane normal to the central longitudinal axis of the expansion and locking mechanism.

Example 56. The implantable prosthetic device of any example herein, particularly any one of examples 49-55, wherein the first and second extension members are shape set into a radially inwardly biased orientation where the free ends extend toward the central longitudinal axis and away from an inner surface of the first member.

Example 57. The implantable prosthetic device of any example herein, particularly any one of examples 49-56, wherein an entirety of the portion of the length of the second member including the plurality of radial extensions is enclosed within the first member.

Example 58. The implantable prosthetic device of any example herein, particularly any one of examples 49-57, wherein the first location is the outflow end portion and the second location is the inflow end portion.

In view of the many possible embodiments to which the principles of the disclosed technology may be applied, it should be recognized that the illustrated embodiments are only preferred examples of the disclosed technology and should not be taken as limiting the scope of the claimed subject matter. Rather, the scope of the claimed subject matter is defined by the following claims and their equivalents.

We claim:

1. An implantable prosthetic device, comprising:
a frame movable between a radially compressed and a radially expanded configuration; and
at least one expansion and locking mechanism comprising:
a first member coupled to the frame at a first location, the first member comprising at least one extension member extending in a circumferential direction and having a free end biased radially inward, into an interior of the first member, relative to a central longitudinal axis of the at least one expansion and locking mechanism; and
a second member coupled to the frame at a second location spaced apart from the first location, the second member extending at least partially inside the first member and comprising a plurality of radial extensions spaced apart from one another along a portion of a length of the second member, wherein the plurality of radial extensions are arranged in a first side of the second member, the first side arranged opposite a second side of the second member, across the central longitudinal axis, the second side not including any radial extensions,
wherein engagement of the at least one extension member with the plurality of radial extensions allows relative movement between the first member and the second member in a first axial direction to allow radial expansion of the frame and prevents relative movement in a second axial direction to prevent radial compression of the frame, and
wherein the second member is rotatable within the first member into a first position where the at least one extension member is biased radially inward and engaged with the plurality of radial extensions and into a second position where the at least one extension member is pushed radially outward and in contact with the second side of the second member.

2. The implantable prosthetic device of claim 1, wherein the plurality of radial extensions is formed by a plurality of radial depressions that extend into an outer surface of the second member, on the first side of the second member.

3. The implantable prosthetic device of claim 2, wherein a radius of the second side of the second member and a radius of each radial extension of the plurality of radial extensions is the same.

4. The implantable prosthetic device of claim 1, wherein in the second position the at least one extension member is disengaged from the plurality of radial extensions and engaged with the second side of the second member to allow relative movement between the first member and the second member in the first axial direction and the second axial direction.

5. The implantable prosthetic device of claim 1, wherein each radial extension of the plurality of radial extensions has an angled edge that is angled relative to a line perpendicular to the central longitudinal axis and an opposing edge that is not angled relative to the line, and wherein the at least one extension member has a corresponding angled edge that is configured to interface with and slide along the angled edge of each radial extension.

6. The implantable prosthetic device of claim 1, wherein the first and second members have a circular cross-sectional profile in a plane normal to the central longitudinal axis of the expansion and locking mechanism.

7. The implantable prosthetic device of claim 1, wherein the at least one extension member is shape set into a radially inwardly biased orientation where the free end extends toward the central longitudinal axis and away from an inner surface of the first member.

8. The implantable prosthetic device of claim 1, wherein an entirety of the portion of the length of the second member including the plurality of radial extensions is enclosed within the first member.

9. The implantable prosthetic device of claim 1, wherein the frame comprises an inflow end portion and an outflow end portion, and wherein the first location is the outflow end portion and the second location is the inflow end portion.

10. The implantable prosthetic device of claim 1, wherein the at least one extension member is a first extension member of the first member, wherein the first member further includes a second extension member spaced axially apart from the first extension member.

11. An assembly, comprising:
a prosthetic valve comprising:
a frame comprising a plurality of interconnected struts and movable between a radially compressed and radially expanded configuration; and
at least one expansion and locking mechanism comprising:
a first member coupled to the frame at a first location, the first member comprising at least one extension member extending in a circumferential direction and having a free end biased radially inward, into an interior of the first member, relative to a central longitudinal axis of the at least one expansion and locking mechanism; and
a second member coupled to the frame at a second location spaced apart from the first location, the second member extending at least partially into the first member and comprising a rack portion including a plurality of spaced apart radial extensions arranged in a first side of the rack portion, the first side arranged opposite a second side of the rack portion that is continuous with a remainder of the second member and does not include radial extensions; and
a delivery apparatus comprising:
a handle;
a first actuation member extending from the handle and coupled to the first member, the first actuation member configured to apply a distally directed force to the first member; and
a second actuation member extending from the handle and coupled to the second member, the second actuation member configured to apply a proximally directed force to the second member,
wherein the prosthetic valve is radially expandable from the radially compressed configuration to the radially expanded configuration upon application of the distally directed force and the proximally directed force to the prosthetic valve via the first and second actuation members, respectively,
wherein expansion of the prosthetic valve causes movement of the first and second members relative to one another such that the at least one extension member engages the first side of the rack portion of the second member, allowing movement of the first and second members in a first direction to allow radial expansion of the frame and preventing movement in a second direction to prevent radial compression of the frame, and
wherein the prosthetic valve is radially compressible from the radially expanded configuration to a less expanded configuration upon rotation of the second member such that the at least one extension member is disengaged from the plurality of radial extensions and engages the second side of the rack portion and allows movement of the first and second members in the first direction and the second direction.

12. The assembly of claim 11, wherein the second member is configured to rotate within the first member and wherein the first member and the second member have circular cross-sections in a plane normal to the central longitudinal axis.

13. The assembly of claim 11, wherein the first member and the second member are concentric with one another and the rack portion of the second member is arranged entirely within the first member.

14. The assembly of claim 11, wherein the plurality of radial extensions are formed by a plurality of radial depressions in the first side of the rack portion, the radial depressions spaced apart from one another along the rack portion of the second member, and wherein a crest of each radial extension of the plurality of radial extensions is arranged in line, in a radial direction, with an outer surface of a remaining portion of the second member, the remaining portion arranged on either side of the rack portion.

15. The assembly of claim 13, wherein each radial extension of the plurality of radial extensions includes an angled side and a non or less angled side, relative to a line arranged perpendicular to the central longitudinal axis, wherein the angled side and the non or less angled side are arranged on opposite sides of a crest of the radial extension.

16. The assembly of claim 15, wherein, during expansion of the prosthetic valve, when the first side of the rack portion is arranged adjacent to and engaged with the at least one extension member, the at least one extension member is configured to slide along the angled side of each radial extension and into a radial depression arranged between two adjacent radial extensions.

17. The assembly of claim 11, wherein the first location is an outflow end portion of the frame and the second location is an inflow end portion of the frame.

18. The assembly of claim 11, wherein the at least one extension member is a first extension member of the first member, wherein the first member further includes a second extension member spaced apart from the first extension member, and wherein the first extension member extends in a first circumferential direction and the second extension member extends in an opposite, second circumferential direction.

19. The assembly of claim 11, wherein the at least one extension member is integral with and formed into the first member and shape set into a radially inwardly biased orientation where the free end extends toward the central longitudinal axis and away from an inner surface of the first member.

20. The assembly of claim 11, wherein the handle includes a control mechanism, actuatable by a user, that actuates relative movement of the first actuation member and the second actuation member.
